(19) United States
(12) Patent Application Publication
(10) Pub. No.: US 2022/0226398 A1
(43) Pub. Date: Jul. 21, 2022

(54) ALGORIPHAGUS SP, BOSEA SP, BREVUNDIMONAS SP, DESULFOVIBRIO SP, MICROBACTERIUM SP, SPHINGOMONAS SP, AND VARIOVORAX SP FOR USE IN DISEASE PREVENTION AND TREATMENT

(71) Applicant: ZIVO Bioscience, Inc., Keego Harbor, MI (US)

(72) Inventors: Amy E. Steffek, Royal Oak, MI (US); William P. Pfund, Kalamazoo, MI (US); Andrew A. Dahl, Bloomfield Hills, MI (US)

(73) Assignee: ZIVO Bioscience, Inc., Keego Harbor, MI (US)

(21) Appl. No.: 17/576,444

(22) Filed: Jan. 14, 2022

Related U.S. Application Data
(60) Provisional application No. 63/137,945, filed on Jan. 15, 2021.

(51) Int. Cl. A61K 35/741; A23L 33/135

(52) U.S. Cl. CPC A61K 35/741; A23L 33/135

(57) ABSTRACT

A treatment compound and its use in the prevention and treatment diseases are disclosed. The compound comprises one or more materials selected from an algal biomass/supernatant (including both algae and bacteria), a bacterial biomass, and isolated and purified compound(s) as well as specific active sites or structures on those compounds. The bacteria are selected from the group consisting of Algoriphagus sp., Bosea sp., Brevundimonas sp., Desulfovibrio sp., Microbacterium sp., Sphingomonas sp., and Variovorax sp. The disclosed inventive compound and method of treatment find particular application in the treatment of disease in poultry, particularly in the prevention and treatment of coccidiosis.

### TECHNICAL FIELD

The present disclosed inventive concept relates to the use of a novel compound in the treatment of various diseases in animals and humans. More particularly, the disclosed inventive concept relates to a method and treatment using a natural feed treatment compound. The treatment compound comprises one or more materials selected from an algal biomass/supernatant (including both algae and bacteria), a bacterial biomass, and isolated and purified compound(s) as well as specific active sites or structures on those compounds. When compared with non-treated animal or human subjects, the compound eliminates or reduces the severity of certain diseases. The compound of the disclosed inventive concept is produced from one or more bacterial strains, many of which are naturally occurring in various environments. The disclosed inventive concept has particular application in the poultry industry but may also find applications beyond poultry to other animals. The disclosed inventive concept may also be beneficial to humans.

### BACKGROUND OF THE INVENTION

The commercial animal industry is under constant economic pressure to develop methods of raising animals that maximize the number of commercially valuable members of a flock or herd. One such industry is the poultry industry which is facing dramatic increases in demand. Poultry meat competes with pork as the world's most consumed meat. It is expected that world poultry production will need to meet an increase in demand of over 120% by the year 2050.

Substantial economic losses in the poultry industry are most often the result of disease. Diseases in flocks often results in reduced production volume or compromised quality of meat. Prevention and treatment of poultry disease adds significantly to poultry production costs. Some estimates place total losses as a result of poultry disease at more than 10% of all production costs.

Of the diseases known to strike poultry flocks, the most common are enteric diseases which include coccidiosis, a disease caused by a parasite, the coccidian protozoa. Annual economic losses due to coccidiosis alone are estimated to exceed $3 billion per year and these costs are expected to increase due to a variety of reasons.

First, coccidiosis prevention today is accomplished mainly through the use of vaccines. A one-time administration of the vaccine is given very early in broiler life and, specifically, on the day of hatch. While this approach has shown some benefit, vaccines are known to suffer from variable effectiveness in controlling the disease over time. Experimentation has shown that a vaccine used in conjunction with a supplement such as a probiotic may improve outcome, but this approach faces its own challenges.

Second, coccidiosis treatment today is accomplished conventionally through the use of antibiotics and ionophores, both of which are costly. The use of antibiotics and ionophores is under pressure globally for a number of reasons, including environmental concerns related to the emergence of antibiotic-resistant pathogens. Drug resistance to antibiotics, ionophores, and synthetic treatment compounds is increasing largely due to overuse thereby severely compromising the effectiveness of these treatments. Relatively recently the European Union banned sub-therapeutic doses of certain antibiotics for use as feed additives. There has been no approval of new drugs in any of these categories for many years. Synthetic treatment compounds and other chemical agents are known but are not as effective as conventional antibiotics.

Third, even if known treatments were still economical and effective, known approaches would still be regarded as unsatisfactory because the medication must be included in the animal's feed for the full duration of its lifespan to be fully effective. This requirement adds significant cost to feed for the entire growout period.

Accordingly, it is desirable to develop a non-antibiotic based treatment of pathogenic infections such as coccidiosis in poultry that is both practical and cost-effective.

### SUMMARY OF THE INVENTION

The disclosed inventive compound and method of treatment relates to a bacteria-based compound for use in the prevention and treatment of a wide variety of diseases, including coccidiosis in poultry. More particularly, the present invention relates to a compound and the use of such a compound such as that derived from a bacterium that selectively alters one or more TLR pathways in the prevention and treatment of disease in both animals and humans. The bacteria are selected from the group consisting of Algoriphagus sp., Bosea sp., Brevundimonas sp., Desulfovibrio sp., Microbacterium sp., Sphingomonas sp., and Variovorax sp.

The compound of the disclosed inventive concept is combined with conventional feed for administration to animals such as poultry for the treatment of disease. The use of the inventive compound disclosed herein may also be used for the treatment of various diseases in humans. The combination of the disclosed inventive compound and conventional feed treats disease conditions by altering one or more TLR pathways. The disclosed inventive compound is a natural product and thus has no adverse environmental impact.

During the treatment period, the disclosed inventive compound is administered to the animal by way of poultry feed, drinking water, or both along with a corn-soy based diet. Studies based on the use of animal feed stock including specific variations of the disclosed inventive compound revealed improved health and disease prevention in animals. Data indicate that feeding chickens (specifically, broiler chickens) a corn/soy diet supplemented with a biomass comprising the inventive compound improves resistance to disease in healthy animals while providing improved treatment in diseased animals. It should be understood that while reference herein is made to a conventional diet of corn and soy, the disclosed compound may also be used to advantage in combination with other forms of conventional animal feed, such as, but not limited to, wheat. Evidence supports the conclusion that the inventive compound alters modulation of the various TLR pathways.

The disclosed inventive concept has numerous advantageous applications in humans and animals including but not limited to: (1) preventing disease in animals, particularly coccidiosis in poultry, (2) providing treatment to diseased animals, particularly in poultry suffering from coccidiosis, and (3) providing both disease prevention and disease treatment in an all-natural compound.

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The method of the disclosed inventive concept proposes the use of a compound in the prevention and treatment of disease in both animals and humans. The compound comprises one or more materials selected from an algal biomass/supernatant (including both algae and bacteria), a bacterial biomass, and isolated and purified compound(s) as well as specific active sites or structures on those compounds. The inventive compound is combined with conventional feed to create a feed mixture that is fed to chickens, for example, broiler chickens, as well as other animals, to both prevent disease in healthy animals and to treat disease in diseased animals.

The Compound Used in Growth Promotion Method and Treatment

By administering the disclosed effective disease prevention and treatment compound early in broiler life, disease may be prevented. By administering the compound to a diseased animal, treatment can be achieved. The effective compound may be derived from the lipopolysaccharide (LPS) layer of a gram-negative bacteria or may be derived from a source other than a lipopolysaccharide.

As used herein, the terms “alteration” or “alter” relate to the impact of a molecule that alters the activity induced by another molecule. By way of example, a compound that might block the LPS-dependent modulation of TLR receptors (including, but not limited to TLR2, TLR3, TLR4, TLR6, TLR7, TLR8, and/or TLR9 receptors) present on the surface of immune cells in humans and animals would be regarded as altering this particular pathway.

As used herein, the term “bacterial culture” is defined as a bacterial organism (one or more types) that grows in a liquid medium. Unless expressly stated otherwise, the term “bacterial biomass” refers to the bacterial cells (with the liquid culture medium removed). The “bacterial biomass” can be wet material or dried material.

Unless expressly stated otherwise, the term “bacterial supernatant” is defined as the culture medium in which the bacterial biomass is grown that contains excreted compounds from the bacterial biomass. Bacterial supernatant is obtained by growing algal biomass in culture medium for an appropriate length of time and then removing the bacterial cells by filtration and/or centrifugation.

Embodiments of the compound used in the growth promotion method and treatment as set forth herein include one or more LPS/Lipid A compounds produced by gram-negative bacterial strains for use in the alteration of one or more of the TLR signaling pathways. The bacterial strains include one or more of the following: Algoriphagus sp., Bosea sp., Brevundimonas sp., Desulfovibrio sp., Microbacterium sp., Sphingomonas sp., and Variovorax sp. Specific species of these bacteria may include one or more of the following: Algoriphagus aquaticus, Algoriphagus aquatilis, Bosea nasdae, Brevundimonas diminuta, Brevundimonas vesicularis, Microbacterium testaceum, and Variovorax paradoxus.

Algoriphagus is a genus of Gram-negative, non-spore-forming, non-motile bacterium found in the biofilm of a freshwater lake.

Bosea is a genus of bacteria from the family of Bradyrhizobiaceae having ten genera and include plant-associated bacteria such as Bradyrhizobium, a genus of rhizobia associated with some legumes.

Brevundimonas is a genus of Proteobacteria, Gram-negative, non-fermenting, aerobic bacilli. The Brevundimonas species are ubiquitous in the environment.

Desulfovibrio is a genus of Gram-negative, sulfate-reducing bacteria and are commonly found in aquatic environments with high levels of organic material, as well as in water-logged soils.

Microbacterium is a genus of Gram-positive endophytic bacterium that resides within plant hosts without causing disease symptoms.

Sphingomonas is a genus of Gram-negative, rod-shaped, chemoheterotrophic, strictly aerobic bacteria.

Variovorax is a genus of Gram-negative aerobic bacterium that can grow under a variety of conditions. It is part of the subclass Proteobacteria and is capable of metabolically utilizing several natural compounds generated by plants or algae.

The disclosed inventive concept involves any combination of two fundamental steps: (1) the gram-negative bacteria produces LPS/Lipid A compounds and (2) the LPS/Lipid compounds modulate TLR activity by altering the signaling pathway thereby preventing or reversing diseases such as coccidiosis. In an embodiment, the LPS/Lipid A compounds produced by the above-noted bacteria used to selectively alter the TLR signaling pathway (including, but not limited to TLR2, TLR3, TLR4, TLR6, TLR7, TLR8, and/or TLR9 receptors). The strains may be naturally occurring and may be found in a bacterial biomass and/or bacterial supernatant products.

The LPS/Lipid A compound employed herein may be obtained from the bacterial strain by any suitable method, but in specific embodiments they are extracted using standard multi-step LPS extraction protocols, such as: (1) extracting freeze-dried bacteria with a solution of phenol/guanidine thiocyanate and collecting the water layer for freeze-drying; (2) resolubilizing the freeze-dried fraction in water; (3) ultrafiltration of the solubilized fraction to remove low molecular weight substances and salts; (4) affinity purifying the high-molecular weight fraction using a polymyxin B resin column such as Affi-prep polymyxin matrix material (Bio-Rad), from which an active fraction is eluted with 1% deoxycholate and, optionally; (5) performing additional purification using size-exclusion chromatography.

### EXAMPLES

Non-limiting examples of a composition and method for preventing and treating disease in animals and humans are set forth. It is to be understood that while the following method is directed to the enhancement of growth in poultry, the disclosed method may apply as well to other animals as well as humans. Accordingly, the described growth promotion method and treatment is not intended as being solely for use in poultry.

According to the present, non-limiting examples, the inventive compound is defined as the bacterial biomass as set forth above and related materials including bacterial supernatant. The inventive compound was mixed with conventional feed to form a supplemented “feed mixture” at a fixed ratio. This ratio was maintained throughout the test period. The bird flock was divided into a control group fed only conventional corn-soy feed and an experimental group fed the supplemented feed mixture.

Specific examples of the bacteria incorporated into the inventive treatment compound include one or more bacteria selected from the consisting of Algoriphagus sp., Bosea sp., Brevundimonas sp., Desulfovibrio sp., Microbacterium sp., Sphingomonas sp., and Variovorax sp. In some examples of the tested compound only a single bacterium was used while in others two or more bacteria were used. For example, in one treatment compound only Variovorax paradoxus was used while in in another Variovorax paradoxus and a combination of species of three bacteria were used.

Four different treatment compounds were batched. In the first two treatment compounds only Variovorax paradoxus was combined with the animal feed with each batch containing different ratios of bacteria to feed. The third treatment compound included four different bacteria, one of which was Variovorax paradoxus. The fourth treatment compound included three different bacteria but excluded Variovorax paradoxus.

Study Procedures

Three control groups and four experimental groups were established. In the first control group the test birds were free of disease, specifically coccidiosis. Accordingly, no treatment was provided to the first control group.

The second control group was challenged with disease, specifically coccidiosis. No treatment was provided to the second control group.

The third control group was also challenged with disease, specifically coccidiosis. This group was treated with a conventional coccidiostat. Specifically, Coban® was provided to the test poultry according to manufacturer's guidelines.

Each of the four experimental groups included coccidiosis infected birds. The first and second experimental groups were treated with the first and second compounds respectively in which only Variovorax paradoxus was combined with the animal feed with each batch containing bacteria produced at different facilities using slightly different conditions.

The third experimental group was treated with the third compound which included four different bacteria, one of which was Variovorax paradoxus. Particularly, the third experimental group included a blend of four bacteria from the genera Variovorax, Sphingomonas, Brevundimonas, and Microbacterium. Both Variovorax and Brevundimonas were provided in the amount of about 45.0 g per ton of the finished feed. Sphingomonas was provided in the amount of 30.0 g per ton of the finished feed. Microbacterium was provided in the amount of about 6.0 g per ton of the finished feed. The total of the four bacteria were provided in the amount of about 126.0 g per ton of feed.

The quantity of the bacteria forming the blend of the third experimental group may be varied. As non-limiting examples, both Variovorax and Brevundimonas may represent between about 30.0 g and 60.0 g per ton of finished feed or more preferably between about 40.0 g and 50.0 g per ton of finished feed, while Sphingomonas may preferably represent between about 15.0 g and 45.0 g per ton of finished feed or more preferably may represent between about 25.0 g and 35.0 g per ton of finished feed, and Microbacterium may represent between about 0.1 g and 20.0 g per ton of finished feed, or more preferably may represent about 1.0 g and 10.0 g per ton of finished feed.

The fourth experimental group was treated with the fourth compound which included three different bacteria excluding Variovorax paradoxus. Particularly, the fourth experimental group included Brevundimonas, Sphingomonas, and Microbacterium. Brevundimonas was provided in the amount of about 45.0 g per ton of finished feed, Sphingomonas was provided in the amount of about 30.0 g per ton of finished feed, and Microbacterium was provided in the amount of about 6.0 g per ton of the finished feed. The total of the three bacteria were provided in the amount of about 81.0 g per ton of feed.

However, it would have been possible to provide different ranges of each bacteria. As non-limiting examples, Brevundimonas may be provided in the amount of between about 30.0 g and 60.0 g per ton of finished feed and more preferably may be provided in the amount of between about 40.0 g and 50.0 g per ton of finished feed, while Sphingomonas may be provided in the amount of between about 15.0 g and 45.0 g per ton of finished feed or more preferably may be provided in the amount of between about 25.0 g and 35.0 g per ton of finished feed, and Microbacterium may be provided in the amount of between about 0.1 g and 20.0 g per ton of finished feed or may most preferably be provided in the amount of between about 1.0 g and 10.0 g per ton of finished feed. Accordingly, the three bacteria defining the biomass may be provided in an amount preferably of between about 60.0 g per ton of finished feed and about 100.0 g per ton of finished feed, and more preferably provided in an amount of between about 70.0 g per ton of finished feed and 90.0 g per ton of finished feed.

The study was undertaken to determine the response and efficacy of a dried algal biomass feed ingredient incorporated at a specific amount into a commercial-type corn-soybean diet and fed to floor-pen raised broilers. The study was undertaken over a 28-day period, from Day 0 to Day 28. Particularly, the treatment compound is fresh water algal biomass containing Gram-negative bacteria provided as animal feed in combination of a feed additive, such as soy oil, preferably though not exclusively at a ratio of two parts soil oil to one part algal biomass. Once the biomass and feed additive are combined to the preferred premix level, the combined batch is poured or administered evenly into a ribbon mixer containing finished feed.

During treatment, the feed conversion ratio (FCR) of each of the test birds was monitored. The morality rate and lesion scores were then identified and recorded.

Two non-limiting embodiments of the biomass provided as animal feed in combination with feed additive are disclosed. The biomass of the first embodiment included a cocktail blend of four bacteria while the biomass of the second embodiment included a blend of three bacteria. A greater or lesser number of bacteria may be included as part of the blend.

Study—Treatment Method

A total of 1,680 mixed sex broiler chicks were obtained from a commercial hatchery on Day 0 (hatch and placement day). A number of mixed-sex broiler chicks (50:50 sex ratio) were randomly assigned on Day 0 by individual weights to one of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study. Bird replicates were 20 with 12 replicates per treatment groups. There were seven treatment groups.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

All birds received nutritionally adequate diets which included algal fermentate of the present composition as pellets and were fed their respective treatment diets ad libitum from day of hatch to 28 days of age. Birds were raised on built-up litter to further mimic stress conditions typically experienced in poultry production.

All diets were offered ad libitum without restrictions to full-fed consumption, except for an 8-hour fasting period prior to cocci-challenge on Day 7 when all birds were challenged by receiving an oocyst-inoculated feed containing a mixture of Eimeria acervulina, Eimeria maxima, and Eimeria tenella. Dietary requirements for protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass).

Cocci-Challenge—On Day 7, all birds received oocyst-inoculated feed containing a mixture of Eimeria acervulina, Eimeria maxima, and Eimeria tenella. Adequate feed was precisely weighed and provided to birds to consume at the rate of 100% fill-capacity on average. Prior to the challenge, all birds were starved for eight hours. Inoculated feed was provided to the birds. Following a specific time, all remaining inoculated feed was removed and weighed to assure equal consumption per pen and per bird. The quantity of feed (both placed and withdrawn) was recorded on each pen's feed record.

Results

In general, analysis of the results appears to support the conclusion that use of the innovative compound in the treatment of coccidiosis-challenged poultry demonstrates a significant improvement in the health of diseased poultry when compared with untreated poultry. The encouraging results below were identified in the different bacterial variations of the disclosed compound.

The results are summarized as follows:

FCR showed improvement in the sample poultry treated with the disclosed composition compared with untreated disease-challenged birds.

Mortality rates of sample poultry treated with the disclosed composition were lower than the mortality rates of untreated disease-challenged birds.

Upon examination of sacrificed sample birds, it was found that the average lesion scores of both the duodenum and the ceca of sample poultry treated with the disclosed composition were lower than the scores of sacrificed untreated disease-challenged birds.

The data demonstrate that when more than one of Algoriphagus sp., Bosea sp., Brevundimonas sp., Desulfovibrio sp., Microbacterium sp., Sphingomonas sp., and Variovorax sp. are included in the compound improvement is seen in FCR, mortality, and lesion scores compared with the untreated disease-challenged birds.

Average body weight of sample poultry treated with the disclosed composition was greater than the average body weight of untreated disease-challenged birds.

The improvement of the overall health of disease-challenged poultry as a result of treatment with the disclosed inventive composition was achieved without the use of antibiotics.

Overall the inventive composition demonstrates a cost-effective and practical approach to the treatment of disease states in animals.

What is claimed is:

1. A composition for the treatment of disease in animals through modulation of the TLR pathway, the composition comprising effective amounts of an algal biomass as an ingredient of finished feed, the algal biomass comprising at least two bacteria selected from the group consisting of Algoriphagus sp., Bosea sp., Brevundimonas sp., Desulfovibrio sp., Microbacterium sp., Sphingomonas sp., and Variovorax sp.

2. A composition for the treatment of disease in animals, the composition including an algal biomass as a portion of finished feed, the algal biomass consisting essentially two or more bacteria selected from the group consisting of Algoriphagus sp., Bosea sp., Brevundimonas sp., Desulfovibrio sp., Microbacterium sp., Sphingomonas sp., and Variovorax sp.

3. The composition of claim 2, wherein the disease being treated is coccidiosis.

4. The composition of claim 2, wherein the algal biomass is fed to the animal in an amount from about 60.0 g per ton of finished feed to about 150.0 g per ton of finished feed.

5. The composition of claim 4, wherein the algal biomass is fed to the animal in an amount from about 60.0 g per ton of finished feed to about 100.0 g per ton of finished feed.

6. The composition of claim 5, wherein the algal biomass includes three bacteria.

7. The composition of claim 6 in which the first bacteria represent between about 40.0 g to about 50.0 g per ton of finished feed, the second bacteria represent between about 25.0 g to about 35.0 g per ton of finished feed, and the third bacteria represent between about 1.0 g and 10.0 g per ton of finished feed.

8. The composition of claim 7 wherein the three bacteria include Brevundimonas sp., Sphingomonas sp., and Microbacterium sp.

9. The composition of claim 8 wherein Brevundimonas sp. comprises the first bacteria, Sphingomonas sp. comprises the second bacteria, and Microbacterium sp comprises the third bacteria.

10. The composition of claim 4, wherein the algal biomass is fed to the animal in an amount from about 100.0 g per ton of finished feed to about 150.0 g per ton of finished feed.

11. The composition of claim 10, wherein the algal biomass is fed to the animal in an amount from about 100.0 g per ton of finished feed to about 150.0 g per ton finished feed.

12. The composition of claim 11, wherein the algal biomass includes four bacteria.

13. The composition of claim 12 in which the first and second bacteria represent between about 40.0 g to about 50.0 g per ton of finished feed, the third bacteria represent between about 25.0 g to about 35.0 g per ton of finished feed, and the fourth bacteria represent between about 1.0 g and 10.0 g per ton of finished feed.

14. The composition of claim 13 wherein the four bacteria include Variovorax sp., Brevundimonas sp., Sphingomonas sp., and Microbacterium sp.

15. The composition of claim 14 wherein Variovorax sp. comprises the first bacteria, Brevundimonas sp. comprises the second bacteria, Sphingomonas sp. comprises the third bacteria, and Microbacterium sp comprises the fourth bacteria.

16. A method for treating an animal for subclinical or clinical coccidiosis through modulation of the TLR pathway, the method comprising administering a biomass-based composition including a compound derived from two or more bacteria at amounts efficacious for the treatment of the subclinical or clinical coccidiosis, the two or more bacteria being selected from the group consisting of Algoriphagus sp., Bosea sp., Brevundimonas sp., Desulfovibrio sp., Microbacterium sp., Sphingomonas sp., and Variovorax sp.

17. The method of claim 16, wherein the biomass-based composition is fed to the animal in an amount from about 60.0 g per ton of finished feed to about 150.0 g per ton of finished feed.

18. The composition of claim 16, wherein the biomass-based composition consists essentially of three bacteria and is fed to the animal in an amount from about 60.0 g per ton of finished feed to about 100.0 g per ton finished feed.

19. The composition of claim 18, wherein the three bacteria include Brevundimonas sp., Sphingomonas sp., and Microbacterium sp.

20. The composition of claim 16 wherein the biomass-based composition consists essentially of four bacteria and is fed to the animal in an amount from about 100.0 g per ton of finished feed to about 150.0 g per ton of finished feed.

21. The composition of claim 20 wherein the four bacteria include Variovorax sp., Brevundimonas sp., Sphingomonas sp., and Microbacterium sp.
